(19) United States
(12) Patent Application Publication
(10) Pub. No.: US 2015/0305824 A1
(43) Pub. Date: Oct. 29, 2015

(54) Technique for Inserting Medical Instruments Using Head-Mounted Display

(71) Applicant: Steven Sounyoung Yu, Rockville, MD (US)

(72) Inventor: Steven Sounyoung Yu, Rockville, MD (US)

(21) Appl. No.: 14/696,466

(22) Filed: Apr. 26, 2015

Related U.S. Application Data
(60) Provisional application No. 61/984,779, filed on Apr. 26, 2014.

(51) Int. Cl. A61B 19/00

(52) U.S. Cl. CPC A61B 19/5244; A61B 2019/5229; A61B 2019/5238; A61B 2019/5276; A61B 2019/262; A61B 2019/5291

(57) ABSTRACT

Techniques for performing image-guided medical procedures using a wearable, head-mounted image display apparatus, which displays images from medical imaging equipment. The clinician views the images provided by the head-mounted image display apparatus to help in inserting medical instruments into a patient's body. Examples of such medical procedures include ultrasound-guided needle/catheter procedures and laryngoscope-guided tracheal intubation. Being able to view the images while simultaneously having a direct view of the procedure site can improve procedure performance. Equipment for performing such procedures include camera laryngoscopes, transesophageal echocardiography apparatus, endoscopic imaging apparatus, x-ray fluoroscope apparatus, or medical ultrasound apparatus.

### TECHNICAL FIELD

My invention relates to medical instruments and medical imaging.

### BACKGROUND

Historically, the expensive cost, limited availability, and bulkiness of medical imaging equipment has prohibited its everyday use in routine clinical practice. More recently, reduced cost and more compact form design has lead to more widespread use of medical imaging technology in routine clinical practice. However, conventional medical imaging technology still relies on bulky display screens to show the images to the clinician. Having the images provided via a head-mounted image display apparatus can improve the way in which medical imaging is used in clinical practice.

### SUMMARY

In one embodiment, my invention is a method of inserting a medical instrument into a patient's body by a percutaneous or per-orifice route. The method comprises wearing a head-mounted image display apparatus that is in communication with a medical imaging equipment. The head-mounted display apparatus provides real-time video images from the medical imaging equipment. The clinician positions the medical instrument at the percutaneous skin site or per-orifice site. The clinician then advances the medical instrument into the patient's body through the skin or orifice site while simultaneously using an external view of the insertion procedure and the real-time video images from the medical imaging equipment to guide the path of the medical instrument into the patient's body. My invention also encompasses medical imaging systems and apparatus for performing the such techniques. Examples include camera laryngoscopes, transesophageal echocardiography apparatus, endoscopic imaging apparatus, x-ray fluoroscope apparatus, and medical ultrasound apparatus.

### BRIEF DESCRIPTION OF THE DRAWINGS

FIG. 1 shows an example of an eyewear image display apparatus that can be used in my invention.

FIG. 2 shows an example of a headband image display apparatus that can be used in my invention.

FIG. 3 shows an example of a transesophageal echocardiography apparatus of my invention.

FIG. 4 shows an example of a gastric endoscope apparatus of my invention.

FIG. 5 shows an example of how my invention could be used for performing a laryngoscope-guided endotracheal intubation on a patient.

FIG. 6 shows another example of how my invention could be used for performing an ultrasound-guided central vein cannulation.

### DETAILED DESCRIPTION

My invention relates to techniques for inserting medical instruments into a patient's body. My invention is useful for image-guided medical procedures (the term “medical procedure” includes surgical procedures) that are not performed with open exposure of the patient's body, such as through a thoracotomy, laparotomy, or craniotomy. Without the visibility provided by open exposure, inserting and properly placing a medical instrument into a patient's body can be challenging. Examples of such medical procedures include those using a percutaneous route (through the skin) or per-orifice route (through a body orifice).

My invention facilitates image-guided medical procedures by using a wearable, head-mounted image display apparatus which produces images for the clinician's view during the medical instrument insertion procedure. A variety of such types of apparatuses are available or have been proposed, and can be used in my invention so long as it is capable of displaying real-time video images from the medical imaging equipment to the user while also simultaneously allowing the user to have a direct view of the real world environment.

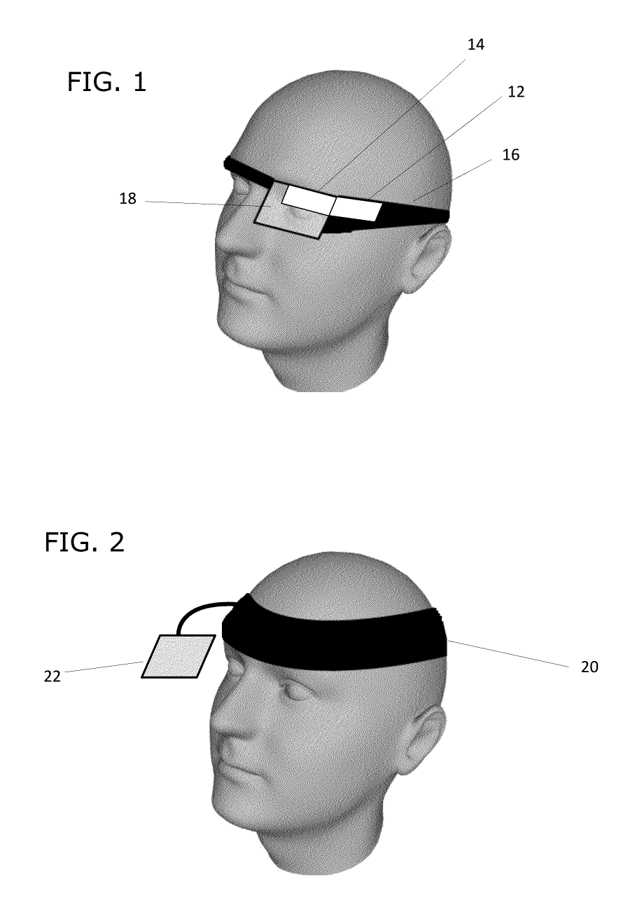

The head-mounted apparatus may be in any suitable form such as eyewear (e.g. glasses or goggles), mask, headband, hat, face shield, etc. For example, FIG. 1 shows an eyewear apparatus 10 that has a small microcomputer device 12 mounted on the eyewear frame 16 and an image projector 14 to project images for viewing by the left eye. The clinician's view from the right eye is unobstructed. Examples of such eyewear apparatus include the Google Glass product and other similar eyewear-type products. In another example as shown in FIG. 2, the apparatus can be a headband 20 with a movable display screen 22 attached for viewing by the right eye whereas the clinician's left eye has an unobstructed view.

The head-mounted apparatus is in communication (wired or wireless) with a medical imaging equipment. The head-mounted apparatus displays images produced by the imaging equipment. My invention can be implemented in a variety of different types of image-guided medical procedures for inserting a variety of different types of medical instruments. The medical instrument may be a component of the medical imaging equipment. In such cases, the medical instrument is coupled (physically or wirelessly) to the medical imaging equipment for signal transmission.

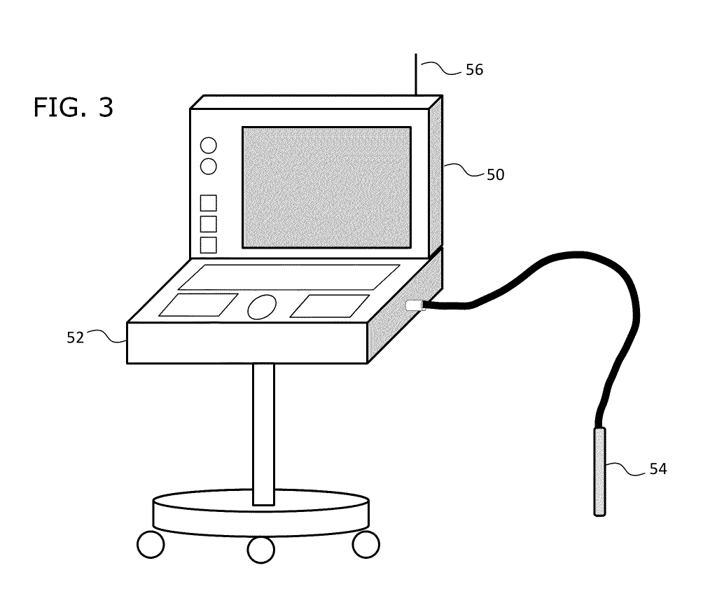

For example, for the purposes of framing my invention, in a transesophageal echocardiography apparatus (medical imaging equipment), the esophageal transducer probe that is inserted into the patient's esophagus may be considered the medical instrument. As an example, FIG. 3 shows a transesophageal echocardiography apparatus that is mounted on a wheeled platform. The esophageal probe 54 is inserted into the patient's esophagus. The esophageal probe 54 is connected to a mobile console 52 that carries a computerized ultrasound image processor, keyboard, track ball, selection buttons, and adjustment knobs. The mobile console 52 also has a display screen 50 to display the ultrasound images and a wireless transmitter with antenna 56 to transmit the ultrasound images to the head-mounted image display apparatus (not shown).

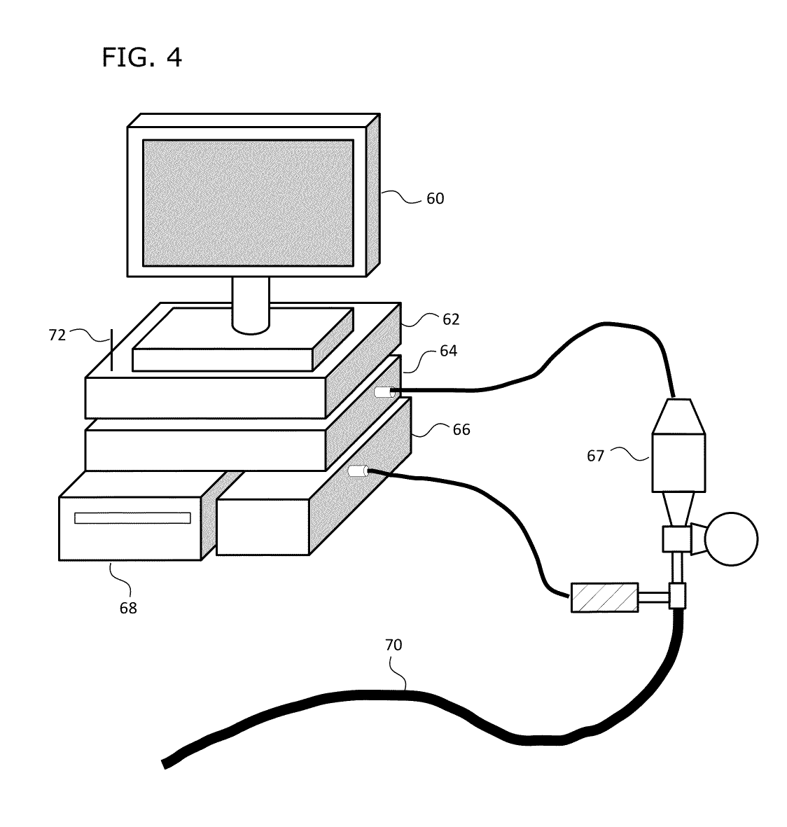

In another example, for the purposes of framing my invention, in an endoscopy apparatus (medical imaging equipment), the endoscope shaft (i.e. the rigid or flexible elongate tubular portion of an endoscope system that contains fiber optic bundles or electrical wiring) that is inserted into the patient's body may be considered the medical instrument. As an example, FIG. 4 shows a gastric endoscope apparatus. The flexible endoscope shaft 70 is for insertion into the patient's esophagus and stomach. The endoscope shaft 70 is coupled to a video camera 67 for capturing the view seen through the endoscope shaft 70. The camera 67 is connected to an endoscopic imaging/processing stack that contains various electronic equipment including video processor 64, light power source 66, display screen 60, printer 68, and computerized control unit 62. This imaging equipment also has a wireless transmitter with antenna 72 to transmit the endoscope images to the head-mounted image display apparatus (not shown). Other types of endoscopes include bronchoscopes, lower gastrointestinal endoscopes such as a sigmoidoscopes, colonsocopes, etc.), ureteroscopes, etc. Some types of endoscopes have a camera at its distal end (i.e. a digital endoscope).

My invention can be implemented with a variety of types of medical instrument designed to be inserted into a patient's body. Examples of medical instruments that may be used include endotracheal tubes, needles, and catheters (long, hollow, and flexible tube designed for insertion into a patient's body for the purpose of withdrawing or infusing a fluid). The medical instrument may be inserted through a percutaneous route (through the skin) or per-orifice route (e.g. mouth, nose, anus, etc.).

In some embodiments, the method is for an image-guided needle or catheter procedure (percutaneous or per-orifice) in which the medical instrument is a needle or catheter. Examples of percutaneous image-guided needle procedures include intravascular catheterization, biopsy (e.g. breast, liver, fine needle aspiration, core biopsy, etc.), aspiration or drainage of fluids (paracentesis, thoracentesis, cyst drainage, abscess drainage, etc.), aspiration or drug injection into spinal vertebra or joints, insertion of electrical neurostimulation leads, drug injections into soft tissue (e.g. corticosteroid or anesthetic injections into ligaments or muscles), perineural injections, and ablation procedures (e.g. by injecting a sclerosing agent). In some cases, the medical imaging equipment is an ultrasound apparatus and the method is for an ultrasound-guided needle or catheter procedure. In some cases, the medical imaging equipment is an x-ray fluoroscope apparatus and the method is for a x-ray fluoroscope guided needle or catheter procedure.

Examples of percutaneous catheter procedures that can be implemented using my invention include intravascular catheterization procedures such as central venous catheterization or arterial catheterization. Examples of central veins include the internal jugular vein in the neck, the subclavian vein in the upper chest, and the femoral vein in the groin. Examples of arterial catheterization include femoral artery catheterization (e.g. for cardiac angiogram) and pulmonary artery catheterization.

My invention may provide various advantages for the clinican. Conventional medical imaging systems often demand that the user keep one eye on the display screen while keeping the other eye on the procedure site. This requires the clinician to turn their head and redirect their gaze back and forth between the display screen and the procedure site. This can cause the clinician to lose focus on what their hands are doing at the procedure site, causing inadvertent movements of the hand and medical instrument when the clinician's head is turned away from the procedure site. Moreover, repeatedly craning their neck back and forth can cause uncomfortable work strain.

In some embodiments, the head-mounted display apparatus provides the video images to only one of the user's eyes (monocular viewing); thus allowing the clinican to have a direct view of the real world environment through the other eye. Simultaneously having an external view of the insertion procedure allows the clinican to have their gaze on the site where the procedure is being performed, while also being able to view the video images from the medical imaging equipment. This can improve procedure performance. This can also allow the clinician to perform the procedure without needing assistance from another person. A variety of types of clinician (e.g. physician, paramedic, nurse, respiratory therapist, paramedic, etc.) may be able to use my invention.

In some embodiments, the medical imaging equipment has a display screen (separate from the head-mounted image display apparatus) and the image resolution provided by the head-mounted image display apparatus is lower than the image resolution provided by the display screen. With the head-mounted image display apparatus providing lower resolution images, the clinician may rely on the head-mounted image display apparatus for inserting the medical instrument. But after the medical instrument is inserted, the clinician may turn to the higher-resolution display screen and rely on it for making a diagnosis (e.g. diagnosing a lesion seen through an endoscope) or performing a therapeutic procedure (e.g. excising a lesion seen through an endoscope). If the clinician no longer needs the image view provided by the head-mounted display apparatus, the clinician can optionally deactivate the head-mounted display apparatus, remove it, push it away to the side, etc.

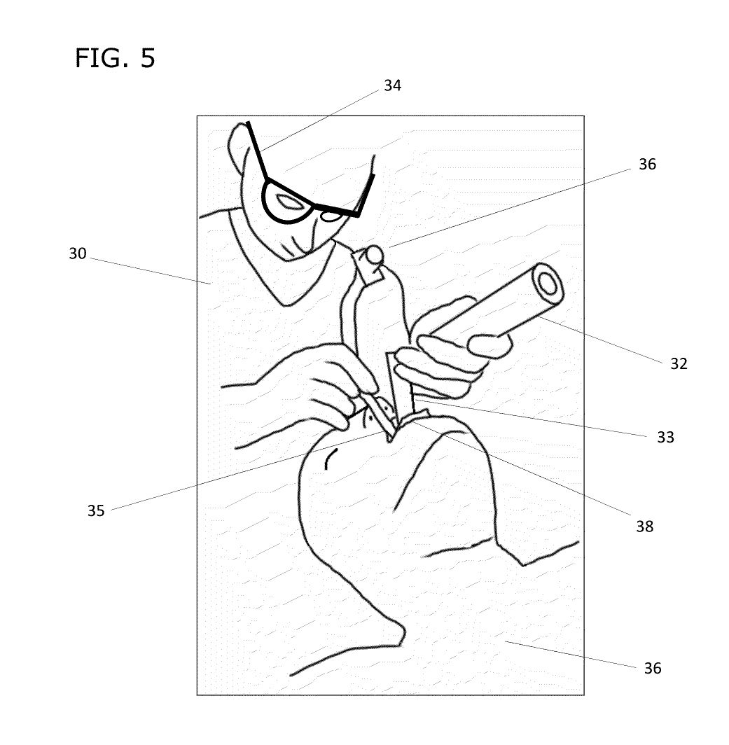

FIG. 5 shows an example of how my invention could be used for performing an endotracheal intubation on a patient 36. The clinician 30 uses a camera laryngoscope (with handle 32 and blade 33) that has a digital camera for viewing the patient's airway (e.g. at or near the distal end of the camera laryngoscope). The camera is in wireless communication with a head-mounted image display apparatus 34 such that it provides real-time video images from the laryngoscope. The clinician 30 wears the head-mounted image display apparatus 34 to see the real-time video images while performing the procedure. The clinician 30 inserts the laryngoscope through the patient's mouth 38 and positions the laryngoscope in the throat to visualize the patient's larynx. The clinician 30 then advances an endotracheal tube (with proximal end 36 and distal end 35) into the patient's trachea while simultaneously relying on an external view of the insertion procedure and the real-time video images from the laryngoscope to guide the path of the endotracheal tube into the patient's trachea.

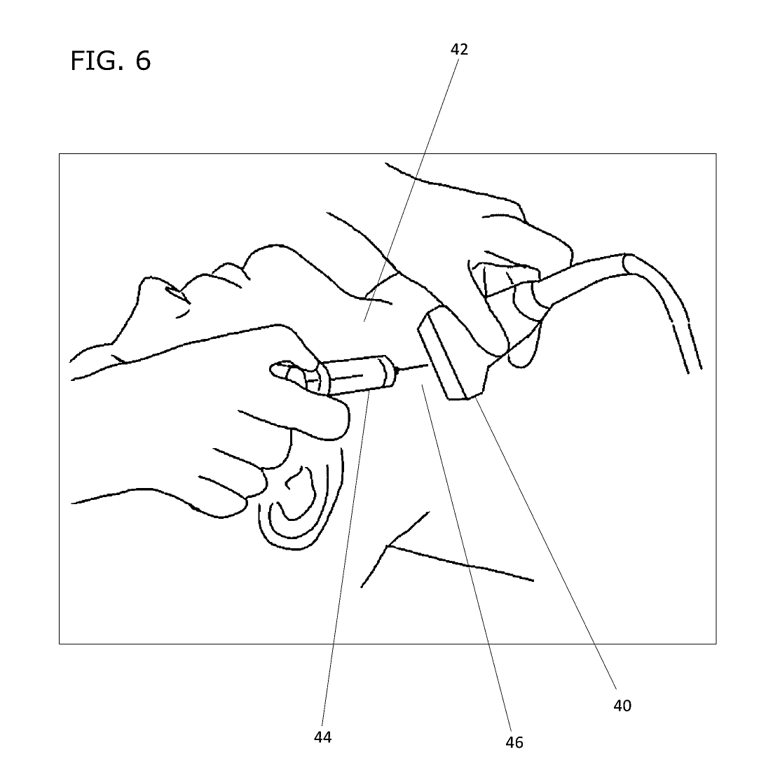

FIG. 6 shows another example of how my invention could be used for performing a central vein cannulation in the patient's neck 42. The clinician uses an ultrasound machine that has a handheld probe 40. The ultrasound machine is in wireless communication with a head-mounted image display apparatus (not shown) such that it provides real-time ultrasound images. The clinician wears the head-mounted image display apparatus to see the real-time ultrasound images while performing the procedure. The clinician finds the internal jugular vein by applying the handheld probe 40 in the area of the target vein. After finding the target vein, the clinician positions a needle 44 at the percutaneous site 46. The clinician then punctures the skin with the needle 44 and advances it into the jugular vein while simultaneously using an external view of the insertion procedure and the real-time ultrasound images to guide the path of the needle into the target vein.

In some embodiments, the method is for an x-ray fluoroscope guided needle procedure, in which the medical imaging equipment is an x-ray fluoroscope and the medical instrument is a needle. In some embodiments, the method is for an endoscopy, in which the medical imaging equipment is an endoscopy imaging apparatus and the medical instrument is an endoscope shaft.

My invention also encompasses medical imaging systems and apparatus for performing the above-described techniques. In one embodiment, my invention is a medical imaging system comprising a medical imaging equipment. The medical imaging equipment comprises a sensor, such as an image capture sensor (e.g. a charge-coupled device or CMOS sensor) or an ultrasound transducer. The medical imaging equipment also comprises a digital image generator coupled to the sensor. The image generator has the processor(s), memory, or other electronic circuitry to produce digital video images from the signal information supplied by the sensor. The medical imaging equipment also has a wireless transmitter coupled to the image generator for transmitting the image information.

The medical imaging system also includes a wearable, head-mounted image display apparatus that is in wireless communication with the wireless transmitter of the medical imaging equipment to display real-time video images from the medical imaging equipment. In some embodiments, the medical imaging equipment also has an attached display screen and the images provided by the head-mounted display apparatus are of lower resolution as compared to the images provided by the attached display screen.

In some embodiments, the medical imaging equipment is a camera laryngoscope comprising an image capture sensor (e.g. a digital camera attached to its distal end as in the configuration of video laryngoscopes that are currently available), a digital image generator connected to the image capture sensor, and a wireless transmitter connected to the image generator. The head-mounted display apparatus is in wireless communication with the wireless transmitter of the laryngoscope. The head-mounted display apparatus provides real-time video images from the laryngoscope. In some cases, the head-mounted image display apparatus provides the video images to only one of the user's eyes.

In some embodiments, the medical imaging equipment is a medical ultrasound apparatus comprising a handheld probe, which contains an ultrasound transducer. The medical ultrasound apparatus further comprises a digital image generator connected to the ultrasound transducer and a wireless transmitter connected to the image generator. The head-mounted image display apparatus is in wireless communication with the wireless transmitter of the medical ultrasound apparatus. The head-mounted display apparatus provides real-time ultrasound images from the medical ultrasound apparatus. In some cases, the head-mounted image display apparatus provides the ultrasound images to only one of the user's eyes. In some cases, the medical ultrasound apparatus has a display screen (separate from the head-mounted display apparatus) and the image provided by the head-mounted display apparatus is of lower resolution than the image provided on the display screen. In some cases, the medical ultrasound apparatus is fully self-contained as a handheld unit and does not include an attached display screen. Examples of such handheld ultrasound units include the handheld probe in the MobiUS ultrasound systems. A self-contained ultrasound apparatus may be particularly advantageous for use with a head-mounted display apparatus because this enhances portability and mobility of the handheld probe. To facilitate mobility, the self-contained handheld unit can be lightweight. In some cases, the fully self-contained handheld unit weighs less than 13.5 ounces.

In some embodiments, the medical imaging equipment is a transesophageal echocardiography apparatus comprising an esophageal probe that is inserted into the patient's esophagus. The esophageal probe contains an ultrasound transducer. The transesophageal echocardiography apparatus further comprises a digital image generator connected to the ultrasound transducer and a wireless transmitter connected to the image generator. The head-mounted image display apparatus is in wireless communication with the wireless transmitter of the transesophageal echocardiography apparatus. The head-mounted display apparatus provides real-time ultrasound images from the transesophageal echocardiography apparatus. In some cases, the head-mounted image display apparatus provides the ultrasound images to only one of the user's eyes. In some cases, the transesophageal echocardiography apparatus has a display screen (separate from the head-mounted display apparatus) and the image provided by the head-mounted display apparatus is of lower resolution than the image provided on the display screen

In some embodiments, the medical imaging equipment is an endoscopic imaging apparatus comprising a digital image generator, a display screen (separate from the head-mounted display apparatus), and a wireless transmitter connected to the digital image generator. The head-mounted image display apparatus is in wireless communication with the wireless transmitter of the endoscopic imaging apparatus. The head-mounted display apparatus provides real-time video images from the endoscopic imaging apparatus. The image provided by the head-mounted display apparatus is of lower resolution than the image provided on the display screen. In some cases, the head-mounted image display apparatus provides the video images to only one of the user's eyes. In some cases, the system further comprises an endoscope connected to the endoscopic imaging apparatus. In some cases, the endoscope comprises an image capture sensor (e.g. in a digital endoscope). In some cases, the endoscopic imaging apparatus comprises an image capture sensor (e.g. a digital camera coupled to a fiber optic endoscope).

In some embodiments, the medical imaging equipment is an x-ray fluoroscope apparatus. The x-ray fluoroscope apparatus comprises an x-ray sensor, a digital image generator connected to the x-ray sensor, and a wireless transmitter connected to the image generator. The x-ray fluoroscope apparatus further comprises a display screen (separate from the head-mounted display apparatus). The head-mounted image display apparatus is in wireless communication with the wireless transmitter of the x-ray fluoroscope apparatus. The head-mounted display apparatus provides real-time video images from the x-ray fluoroscope apparatus. The image provided by the head-mounted display apparatus is of lower resolution than the image provided on the display screen. In some cases, the head-mounted image display apparatus provides the video images to only one of the user's eyes.

The foregoing description and examples have been set forth merely to illustrate my invention and are not intended to be limiting. Each of the disclosed aspects and embodiments of my invention may be considered individually or in combination with other aspects, embodiments, and variations of my invention. In addition, unless otherwise specified, the steps of the methods of my invention are not confined to any particular order of performance. Modifications of the disclosed embodiments incorporating the spirit and substance of my invention may occur to persons skilled in the art, and such modifications are within the scope of my invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

I claim:

1. In a medical procedure, a method of inserting a medical instrument into a patient's body by a percutaneous or per-orifice route, comprising:
wearing a head-mounted image display apparatus that is in communication with a medical imaging equipment, wherein the display apparatus provides real-time video images from the medical imaging equipment;
positioning the medical instrument at the percutaneous skin site or per-orifice site;
advancing the medical instrument into the patient's body through the skin or orifice site while simultaneously using an external view of the insertion procedure and the real-time video images from the medical imaging equipment to guide the path of the medical instrument into the patient's body.

2. The method of claim 1, wherein the medical procedure is a percutaneous ultrasound-guided needle procedure and the medical instrument is a needle.

3. The method of claim 1, wherein the medical procedure is a percutaneous x-ray fluoroscope-guided needle procedure and the medical instrument is a needle.

4. The method of claim 1, wherein the medical procedure is a percutaneous ultrasound-guided catheterization procedure and the medical instrument is a catheter.

5. The method of claim 1, wherein the medical procedure is a percutaneous x-ray fluoroscope-guided catheterization procedure and the medical instrument is a catheter.

6. The method of claim 1, wherein the medical procedure is a transesophageal echocardiography and the medical instrument is an esophageal ultrasound probe.

7. The method of claim 1, wherein the medical procedure is an endoscopy and the medical instrument is an endoscope.

8. The method of claim 1, wherein the medical procedure is tracheal intubation;
wherein the medical instrument is an endotracheal tube; and
wherein the medical imaging equipment is a camera laryngoscope.

9. The method of claim 8, wherein the head-mounted image display apparatus provides the video images to only one of the user's eyes.

10. The method of claim 1, wherein the medical procedure is an ultrasound-guided procedure and the medical imaging equipment is a fully self-contained handheld ultrasound machine without any attached display screen.

11. The method of claim 10, wherein the head-mounted image display apparatus provides the video images to only one of the user's eyes.

12. The method of claim 11, wherein the medical procedure is an ultrasound-guided needle or catheter procedure and the medical instrument is a needle or catheter.

13. The method of claim 12, wherein the self-contained handheld ultrasound machine weighs less than 13.5 ounces.

14. The method of claim 1, wherein the medical imaging equipment has a display screen;
wherein the image resolution provided by the head-mounted image display apparatus is lower than the image resolution provided by the display screen; and
wherein the user turns to view the display screen after inserting medical instrument.

15. The method of claim 1, wherein the medical instrument is an endotracheal tube, the medical imaging equipment is a camera laryngoscope, the medical procedure is an endotracheal intubation, and the method is for inserting the endotracheal tube into a patient using the laryngoscope, the method comprising:
wearing the head-mounted image display apparatus that is in communication with the laryngoscope, wherein the display apparatus provides real-time video images from the laryngoscope;
inserting the laryngoscope through the patient's mouth and positioning it to view the patient's airway;
positioning the endotracheal tube at the patient's mouth; and
advancing the endotracheal tube into the patient's airway while simultaneously using an external view of the insertion procedure and the real-time video images from the laryngoscope to guide the path of the endotracheal tube into the patient's airway.

16. The method of claim 15, wherein the head-mounted image display apparatus provides the video images to only one of the user's eyes.

17. The method of claim 1, wherein the medical imaging equipment is an ultrasound apparatus having a display screen;
wherein the image resolution provided by the head-mounted image display apparatus is lower than the image resolution provided by the display screen; and
wherein the head-mounted image display apparatus provides the video images to only one of the user's eyes.

18. The method of claim 1, wherein the medical procedure is a transesophageal echocardiography, the medical instrument is an esophageal ultrasound probe, the medical imaging equipment is a transesophageal echocardiography apparatus attached to the esophageal ultrasound probe and having a display screen, the method comprising:
wearing the head-mounted image display apparatus that is in communication with the transesophageal echocardiography apparatus, wherein the display apparatus provides real-time video images from the transesophageal echocardiography apparatus;
inserting the esophageal probe through the patient's mouth and into the esophagus while simultaneously using an external view of the insertion procedure and the real-time ultrasound images from the transesophageal echocardiography apparatus to guide the path of the esophageal probe.

19. The method of claim 18, wherein the head-mounted image display apparatus provides the video images to only one of the user's eyes.

20. The method of claim 1, wherein the medical procedure is an x-ray fluoroscope-guided needle or catheterization procedure;
wherein the medical instrument is a needle or catheter; and
wherein medical imaging equipment is an x-ray fluoroscope apparatus.
